(19) United States
(12) Patent Application Publication
(10) Pub. No.: US 2023/0019849 A1
(43) Pub. Date: Jan. 19, 2023

(54) Method for Preparing CD7-Negative, CD3-Positive T Cells

(71) Applicant: ST. JUDE CHILDREN'S RESEARCH HOSPITAL, INC., Memphis, TN (US)

(72) Inventors: Stephan GOTTSCHALK, Memphis, TN (US); Mireya VELASQUEZ, Memphis, TN (US); Abdullah FREIWAN, Memphis, TN (US); Abishek VAIDYA, Memphis, TN (US); Deanna LANGFITT, Memphis, TN (US)

(21) Appl. No.: 17/783,040

(22) PCT Filed: Dec. 4, 2020
(86) PCT No.: PCT/US2020/063259

§ 371 Date: Jun. 7, 2022

Related U.S. Application Data
(60) Provisional application No. 62/945,267, filed on Dec. 9, 2019.

(51) Int. Cl. C12N 5/0783; C12N 5/00; B01D 15/38

(52) U.S. Cl. CPC C12N 5/0636; C12N 5/0087; B01D 15/3809; C12N 2510/00; C12N 2501/2307; C12N 2501/2315

(57) ABSTRACT

Methods for preparing CD7-negative, CD3-positive T cells, which optionally express a chimeric antigen receptor, are provided as is a method of using such cells in a method for treating cancer, in particular a CD7+ cancer. In one aspect, the invention provides a method for preparing a population of CD7-negative, CD3-positive T cells by (a) performing a first selection by depleting, from a population of primary immune cells, cells that express CD7 thereby generating a population of CD7-negative cells; (b) performing a second selection by enriching, from the population of CD7-negative cells, T cells that express CD3 thereby generating a population of CD7-negative and CD3-positive T cells, and (c) incubating the population of CD7-negative and CD3-positive T cells in a culture vessel under stimulating conditions, thereby generating stimulated CD7-negative, CD3-positive T cells.

### INTRODUCTION

This application claims priority to U.S. Provisional Application No. 62/945,267, filed Dec. 9, 2019, the content of which is incorporated herein by reference in its entirety.

This invention was made with government support under grant no. CA021765, awarded by the National Institutes of Health. The government has certain rights in this invention.

### BACKGROUND

Relapsed/refractory hematological malignancies are challenging diseases with poor prognosis. One approach to treating these patients is to genetically modify T cells to target antigens expressed on tumor cells through the expression of chimeric antigen receptors (CARs). CARs are antigen receptors that are designed to recognize cell surface antigens in a human leukocyte antigen-independent manner. CARs have shown promising results in CD19+ malignancies as exemplified in Brentjens, et al. (2010) Mol. Ther. 18:4, 666-668; Morgan, et al. (2010) Mol. Ther. 18:843-851; Till, et al. (2008) Blood 112:2261-2271; Maude, et al. (2014) N. Engl. J. Med. 371(16):1507-17; Maude (2018) Clin. Adv. Hematol. Oncol. 16(1):664-6; Locke, et al. (2017) Mol. Ther. 25:285-95; Lee, et al. (2015) Lancet 385:517-28; Kochenderfer, et al. (2015) J. Clin. Oncol. 33:540-9; Davila, et al. (2014) Sci. Transl. Med. 6:224-225; Gardner, et al. (2017) Blood 129:3322-31. Finding a targetable antigen for T-ALL, however, has been challenging due to the absence of a widely expressed antigen on T-ALL and the concomitant expression on normal activated T cells.

CD7 has emerged as a promising target for the adoptive immunotherapy with T cells expressing chimeric antigen receptors (CAR T cells) of CD7⁺ T cell acute lymphoblastic leukemia (T-ALL) and acute myeloid leukemia (AML). However, expressing CD7 CARs in T cells results in fratricide. Strategies have been developed to overcome this limitation by additional genetic modifications of CD7 CAR T cells. In addition, US 2019/0144522 A1 describes a method for producing anti-CD7 CAR T cells, wherein the method includes the steps of obtaining a population of CD7-negative T cells and transducing the CD7-negative T cells with a nucleic acid construct encoding an anti-CD7 CAR. However, improved methods for isolating and using naturally occurring CD7-negative (CD7⁻) T cells for the adoptive immunotherapy are needed.

### SUMMARY OF THE INVENTION

In one aspect, the invention provides a method for preparing a population of CD7-negative, CD3-positive T cells by (a) performing a first selection by depleting, from a population of primary immune cells, cells that express CD7 thereby generating a population of CD7-negative cells; (b) performing a second selection by enriching, from the population of CD7-negative cells, T cells that express CD3 thereby generating a population of CD7-negative and CD3-positive T cells, and (c) incubating the population of CD7-negative and CD3-positive T cells in a culture vessel under stimulating conditions, thereby generating stimulated CD7-negative, CD3-positive T cells.

In another aspect, this invention provides a method for preparing genetically modified CD7-negative, CD3-positive T cells by (a) performing a first selection by depleting, from a population of primary immune cells, cells that express CD7 thereby generating a population of CD7-negative cells; and (b) performing a second selection by enriching, from the population of CD7-negative cells, T cells that express CD3 thereby generating a population of CD7-negative and CD3-positive T cells, (c) incubating the population of CD7-negative and CD3-positive T cells in a culture vessel under stimulating conditions, thereby generating stimulated T cells; and (d) genetically modifying the stimulated T cells generated in (c), e.g., introducing nucleic acids encoding a CAR into the T cells. In some embodiments, the first, second, or first and second selection includes the use of immunoaffinity-based selection, e.g., an antibody immobilized on or attached to an affinity chromatography matrix, magnetic particle or label.

In a particular aspect, the invention provides a method for preparing CD7-negative, CD3-positive, chimeric antigen receptor T cells by (a) performing a first selection by depleting, from a population of primary immune cells, cells that express CD7 by (i) contacting the population of primary human immune cells with a first immunoaffinity reagent that specifically binds to CD7 on the surface of the primary human immune cells; and (ii) recovering cells of the population that are not bound to the first immunoaffinity reagent thereby generating a population of CD7-negative cells; (b) performing a second selection by enriching, from the population of CD7-negative cells, T cells that express CD3 by (i) contacting the population of CD7-negative cells with a second immunoaffinity reagent that specifically binds to CD3 on the surface of the CD7-negative cells; (ii) recovering T cells of the population that are bound to the second immunoaffinity reagent thereby generating a population of CD7-negative and CD3-positive T cells, (c) incubating the population of CD7-negative and CD3-positive T cells in a culture vessel under stimulating conditions, thereby generating stimulated T cells; and (d) introducing a genetically engineered antigen receptor into the stimulated T cells generated in (c).

Populations of CD7-negative, CD3-positive T cells prepared by the methods of the invention are also provided, as is a method of using such cells in a method for treating cancer, e.g., a CD7⁺ cancer.

### BRIEF DESCRIPTION OF THE DRAWINGS

FIG. 1 shows the 2-step magnetic bead depletion/selection procedure of the present invention.

FIG. 2 is a schematic showing the steps used in the preparation and characterization of the CD7⁻ CD3⁺ T cells.

FIG. 3 shows that compared to non-transduced bulk T cells (NT^{bulk}), CD7⁻ CD⁺ T cells transduced (CD7 CAR) or non-transduced (NT) with a CD7 CAR are composed predominantly of CD4⁺ cells by day 7 (N=11).

FIG. 4 shows that CD7⁻ CD3⁺ T cells (NT^{CD7-} and CD7 CAR^{CD7-} T cells) exhibit a predominantly memory phenotype by day 7 as determined by CCR7 and CD45RA expression. Naïve (CCR7⁺/CD45RA⁺), central memory (CM, CCR7⁺/CD45RA⁻), effector memory (EM, CCR7⁻/CD45RA⁻), and terminal effector (EMRA, CCR7⁻/CD45RA⁺). N=12.

FIG. 5 shows that CD7 CAR^{CD7-} T cells recognize CD7⁺ targets in vitro as demonstrated by a high level of IFNγ production in the presence of CD7⁺ targets (CCRF) but not in the presence of CD7⁻ targets (BV173) or media alone (N=9 P<0.0001).

FIG. 6 shows that CD7 CAR^{CD7-} T cells kill CD7⁺ targets in vitro as demonstrated by tumor cell lysis with a luciferase-based cytotoxicity assay. CD7 CAR^{CD7-} T cells lysed CD7⁺ (CCRF) but not CD7⁻ (Daudi.ffluc) targets at different effector: target (E:T) ratios. (N=3; *p=0.05, ** p=0.01, *** p=0.001, **** p=0.0001).

FIG. 7 is a Kaplan-Meier survival curve showing survival advantage of animals injected with CCRF.ffluc tumor cells and treated with CD7 CAR^{CD7-} T cells on day 7 (median survival tumor only: 20.5 days, CD7 CAR^{CD7-}: 70 days, N=10 P<0.0001).

FIG. 8 shows persistence of CD7 CAR+ T cells as determined by flow cytometry evaluation of peripheral blood 104 days after injection.

FIG. 9 is a Kaplan-Meier survival curve showing survival advantage of animals injected with 3×10⁶ Bv173.ffluc tumor cells and treated with CD19 CAR^{CD7-} T cells on day 7.

### DETAILED DESCRIPTION OF THE INVENTION

A method to select naturally occurring CD7-negative CD3-positive T cells and genetically modifying the same, e.g., to express a CAR, has now been developed. The selection steps include a two-step process (CD7⁻ and CD3⁺ selection) using magnetic beads and subsequently genetically modifying these cells. Notably, a genetically modified CD7-negative CD3-positive T cell population expressing CARs such as CD7 CAR or CD19 CAR constructs exhibit a predominantly CD4⁺ effector memory phenotype, and potent anti-leukemia activity in vitro and in vivo. Thus, naturally occurring CD7⁻ CD3⁺ T cells provide a useful T cell source for the cellular immunotherapy of cancer, in particular CD7⁺ leukemia.

According, the invention provides methods for preparing CD7-negative, CD3-positive T cells and optionally genetically modifying the same. The methods of the invention include the steps of (a) performing a first selection by depleting, from a population of primary immune cells, cells that express CD7 thereby generating a population of CD7-negative cells; (b) performing a second selection by enriching, from the population of CD7-negative cells, T cells that express CD3 thereby generating a selected population of CD7-negative and CD3-positive T cells, (c) incubating the population of CD7-negative and CD3-positive T cells in a culture vessel under stimulating conditions, thereby generating stimulated T cells; and optionally (d) genetically modifying the stimulated T cells generated in (c), e.g., by introducing or deleting a gene of interest.

As used herein, a statement that a cell or population of cells is “negative” for a particular marker refers to the absence of substantial detectable presence on or in the cell of a particular marker, typically a surface marker. In accordance with the present invention, the method and compositions herein are for providing CD7-negative cells. In this respect, a “CD7-negative” or “CD7⁻” cell refers to the absence of substantial detectable presence of CD7 on or in the cell.

As used herein, a statement that a cell or population of cells is “positive” for a particular marker refers to the detectable presence on or in the cell of a particular marker, typically a surface marker. In accordance with the present invention, the method and compositions herein are for providing CD3-positive cells. In this respect, a “CD3-positive” or “CD3+” cell refers to the detectable presence of CD3 on or in the cell.

When referring to a surface marker, the term refers to the absence or presence of surface expression as detected by flow cytometry, for example, by staining with an antibody that specifically binds to the marker and detecting said antibody. In instances where the surface marker is absent, staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker. In instances where the surface marker is present, staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

CD7⁻ CD3⁺ T cells of the invention are obtained by performing a selection, isolation and/or enrichment of a cell sample, such as a primary human immune cell sample or population of primary T cells. Among samples that include a population of primary human immune cells are blood and blood-derived samples, such as white blood cell samples, apheresis samples, leukapheresis samples, peripheral blood mononuclear cell (PBMC) samples, and whole blood. Such samples are known in the art to include T cells, B cells, NK cells and monocytes.

As a first step of the claimed method, cells that express CD7 are depleted from the population of primary human T cells. As used herein, “depleting” when referring to one or more particular cell types or cell population, refers to decreasing the number or percentage of the cell type or population, e.g., compared to the total number of cells in or volume of the composition, or relative to other cell types, such as by negative selection based on markers expressed by the population or cell, or by positive selection based on a marker not present on the cell population or cell to be depleted. The term preferably requires removal of a substantial number of the cell, cell type, or population from the composition.

In certain aspects of this invention, the first selection step includes immunoaffinity-based selection. In accordance with this aspect, immunoaffinity-based selection includes the use of an antibody, i.e., an anti-CD7 antibody, that selectively binds CD7 expressed on the surface of cells of the population of primary T cells. Upon binding between the cell surface localized CD7 and the anti-CD7 antibody, the CD7-expressing cells (i.e., CD7⁺ cells) can be removed or separated from cells that do not express CD7 (i.e., CD7⁻ cells). In some embodiments, the anti-CD7 antibody is immobilized on or attached to an affinity chromatography matrix or support such as a sphere or bead (e.g., microbead, nanobead, including agarose), a magnetic particle (e.g., magnetic bead or paramagnetic bead) or label (e.g., biotin, epitope tag, etc.) to allow for separation of the CD7⁺ cells from CD7⁻ cells. In certain embodiments, the anti-CD7 antibody is attached to a label, in particular biotin. In accordance with this embodiment, the anti-CD7 antibody is covalently attached to the biotin and depletion of CD7-expressing cells is affected by contacting cells bound by the biotinylated anti-CD7 antibody with a reagent that binds biotin, e.g., an immobilized anti-biotin antibody (e.g., an anti-biotin microbead or magnetic bead) or immobilized streptavidin (e.g. a streptavidin microbead or magnetic bead) to allow for separation of the CD7⁺ cells from CD7⁻ cells.

As a second step of the claimed method, cells that express CD3 are enriched for from the population of CD7⁻ negative cells. As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term preferably requires that the cells so enriched be present at or near 100% (e.g., 90%, 92%, 94%, 96%, or 98%) in the enriched population.

In certain aspects of this invention, the second selection step includes immunoaffinity-based selection. In accordance with this aspect, immunoaffinity-based selection includes the use of an antibody, i.e., an anti-CD3 antibody, that selectively binds CD3 expressed on the surface of cells of the population of CD7⁻ negative cells obtained from the first selection step. Upon binding between the cell surface localized CD3 and the anti-CD3 antibody, the CD3-expressing cells (i.e., CD3+ cells) can be removed or separated from cells that do not express CD3 (i.e., CD3⁻ cells). In some embodiments, the anti-CD3 antibody is immobilized on or attached to an affinity chromatography matrix or support such as a sphere or bead (e.g., microbead, nanobead, including agarose), a magnetic particle (e.g., magnetic bead or paramagnetic bead) or label (e.g., biotin, epitope tag, etc.) to allow for separation of the CD3⁺ cells from CD3⁻ cells. In certain embodiments, the anti-CD3 antibody is attached to a magnetic particle. In accordance with this embodiment, the anti-CD3 antibody is covalently attached to a magnetic particle and enrichment of CD3-expressing cells is affected by contacting cells bound by the anti-CD3 antibody-magnetic bead with a magnet to allow for separation of the CD3⁺ cells from CD3⁻ cells, i.e., retention of CD3⁺ cells and elution of CD3⁻ cells. Subsequent elution of the CD3⁺ cells is achieved by removal of the magnet.

The selection steps are generally carried out under conditions suitable to allow binding of the antibodies to cell surface molecules, if present on cells within the sample. In some embodiments, such conditions include a saline solution, pH and temperature conducive to antibody protein interactions. By way of illustration, selection occurs at 4° C. in phosphate-buffered saline at pH 7-8.

In some embodiments, the spheres or beads (including (e.g., magnetic beads or paramagnetic beads) used in the above-referenced separation steps can be packed into a column to effect immunoaffinity chromatography, in which a sample containing cells (e.g., primary human T cells containing CD7⁺ cells, or CD7⁻ cell containing CD3⁺ cells) is contacted with the matrix of the column and subsequently eluted or released therefrom. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. II, Brooks & Schumacher (eds), p 17-25, Humana Press Inc., Totowa, N.J.). Such beads are known and are commercially available from a variety of sources including, in some aspects, magnetic beads sold under the tradenames DYNABEADS® (Life Technologies, Carlsbad, Calif.), MACS® beads (Miltenyi Biotec, San Diego, Calif.) or STREPTAMER® beads (IBA, Germany).

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed in accordance with the present invention, where negative fractions are retained in the first selection step and positive fractions are retained in the second selection step.

In some embodiments, the magnetically responsive particles are left attached to the cells that are to be subsequently incubated, cultured and/or engineered. In some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (Miltenyi Biotech, Auburn, Calif.). Magnetic-activated cell sorting systems are capable of high-purity selection of cells having magnetized particles attached thereto. In certain embodiments, magnetic-activated cell sorting operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered.

Using the two-step selection method of the invention, a selected population of CD7-negative and CD3-positive T cells is obtained. These T cells are characterized as lacking cell surface expression of CD7, exhibiting cell surface expression of CD3⁺. In some embodiments, the yield of the CD7⁻ CD3⁺ T cells in the selected population of cells, i.e., the number of cells in the population or sub-population compared to the number of the same population or sub-population of cells in the starting sample, is 10% to 100%, such as 20% to 80%, 20% to 60%, 20% to 40%, 40% to 80%, 40% to 60%, or 60%, to 80%. In some embodiments, the yield of the CD7⁻ CD3⁺ T cells in the selected population of cells is less than 70%, less than 60%, less than 50%, less than 40%, less than 30% or less than 20%.

In some embodiments, the purity of the CD7⁻ CD3⁺ T cells in the selected population of cells, i.e., the percentage of cells negative for CD7 and positive for CD3 versus total cells in the selected population of cells, is at least 90%, 91%, 92%, 93%, 94%, and is generally at least 95%, 96%, 97%, 98%, 99% or greater.

The CD7⁻ CD3⁺ T cells thus obtained are generally maintained in a vessel, e.g., a tube, tubing set, chamber, unit, well, culture vessel, and/or bag under suitable culture conditions to maintain viability, proliferative capacity and function. Accordingly, the next step of the method of this invention provides for incubating the population of CD7⁻ CD3⁺ T cells in a culture vessel under stimulating conditions, thereby generating stimulated cells. In some aspects, the stimulating conditions for the incubation include conditions whereby CD7⁻ CD3⁺ T cells proliferate or expand. For example, in some aspects, the incubation is carried out in the presence of an agent capable of activating one or more intracellular signaling domains of one or more components of a TCR complex, such as a CD3 zeta chain, or capable of activating signaling through such a complex or component. In some aspects, the incubation is carried out in the presence of an anti-CD3 antibody, and anti-CD28 antibody, anti-4-1BB antibody, for example, such antibodies coupled to or present on the surface of a solid support, such as a bead; and/or a cytokine, such as IL-2, IL-15, IL-7, and/or IL-21. Ideally, stimulating conditions maintains viability, proliferative capacity and function of the cells and enhances the expansion of memory T cells. Thus, in certain embodiments, the CD7⁻ CD3⁺ T cells exhibit a predominantly CD4⁺ memory phenotype as evidenced by, e.g., CD4, CCR7 and CD45RA expression. In this respect, the method of this invention is also recognized as a method for providing for CD7⁻ CD3⁺ CD4⁺ memory T cells. In particular embodiments, CD7⁻ CD3⁺ CD4⁺ memory T cells include effector memory cells. In some aspects, a population of cells enriched for effector memory T cells exhibits the phenotype of being CCR7-negative (CCR7⁻) and CD45RA-negative (CD45RA⁻).

In some embodiments, the population of primary immune cells is obtained from a subject. In some aspects, the subject from whom the population of primary immune cells is obtained is a subject to be treated with the selected CD7⁻ CD3⁺ T cells. In other aspects, the subject from whom the population of primary immune cells is obtained is a subject other than the subject to be treated with the selected CD7⁻ CD3⁺ T cells. In accordance with either aspect, the method of the invention can also provide for genetically engineering the stimulated CD7⁻ CD3⁺ T cells for adoptive cell therapy. Thus, the inventive method may further include the step of genetically modifying the stimulated CD7⁻ CD3⁺ T cells.

T cells that are “genetically modified” refer to T cells that have been manipulated by recombinant methods. In some aspects, the genetically modified T cells are genetically modified by removing, deleting or modifying one or more genes of interest of the genome of the T cells. In other aspects, the genetically modified T cells are genetically modified by introducing one or more genes of interest into the T cells. In some aspects, the T cells are genetically modified to express an engineered antigen receptor. In this respect, the methods of the invention can further include the step of introducing into the stimulated T cells a construct harboring nucleic acids encoding an engineered antigen receptor.

In one aspect, the engineered antigen receptor is a chimeric antigen receptor. A “chimeric antigen receptor,” “CAR,” “chimeric immunoreceptor,” “chimeric T cell receptor,” or “artificial T cell receptor” is a receptor protein that has been engineered to give T cells the ability to target a specific protein. The receptors are chimeric because they combine both antigen-binding and T cell activating functions into a single receptor. CARs generally include an extracellular ligand binding domain linked to one or more intracellular signaling components. Such molecules typically mimic or approximate a signal through a natural antigen receptor and/or signal through such a receptor in combination with a costimulatory receptor. Preferably, the CAR specifically binds to a ligand on a cell or disease to be targeted, such as a cancer or other disease or condition, including those described herein for targeting with the provided methods and compositions. Exemplary antigens are orphan tyrosine kinase receptor EGFR (Epidermal Growth Factor Receptor), EGFRvIII (Epidermal Growth Factor Receptor Variant III), Her2 (Human Epidermal Growth Factor Receptor 2), FR-α (Folate Receptor Alpha), L1-CAM (L1 cell adhesion molecule), CAIX (Carbonic anhydrase 9), CD7 (Cluster of differentiation 7), CD19, CD20, CD22, mesothelin, CEA (Carcinoembryonic Antigen), and hepatitis B surface antigen, CD23, CD24, CD30, CD33, CD38, CD44, EGP-2 (epithelial glycoprotein 2), EGP-4, EPHa2 (ephrin type-A receptor 2), ErbB2 (v-erb-b2 erythroblastic leukemia viral oncogene homolog 2), ErbB3, ErbB4, FBP (folate binding protein), AChR (fetal acetylcholine receptor), GD2 (Disialoganglioside GD2), GD3, GPC3 (Glypican-3), HMW-MAA (high molecular weight melanoma-associated antigen), IL22-Rα (Interleukin-22 Receptor alpha), IL13-Rα2, IL13-Rα3, kappa light chain, Lewis Y, MAGE-A1 (Melanoma-associated antigen-A1), MAGE-A3, MUC1 (Mucin-1), MUC16, PSCA (prostate stem cell antigen), PSMA (Prostate-specific membrane antigen), NKG2D (natural killer group 2 member D) ligands, NY-ESO-1 (New York Esophageal Squamous Cell Carcinoma-1), MART-1 (Melan-A), gp100, oncofetal antigen, ROR1 (Receptor Tyrosine Kinase Like Orphan Receptor 1), TAG72 (Tumor-associated glycoprotein 72), VEGFR2 (Vascular endothelial growth factor receptor 2), carcinoembryonic antigen (CEA), estrogen receptor, progesterone receptor, ephrinB2, CD123, CS-1, c-Met, and/or molecules expressed by HIV, HCV, HBV or other pathogens.

To target one or more of the above-reference antigens, CARs include in their extracellular portion one or more antigen binding molecules, such as one or more antigen-binding fragments, domains, or portions, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the CAR includes an antibody heavy chain domain that specifically binds a cell surface antigen of a cell or disease to be targeted, such as a tumor cell or a cancer cell, such as any of the target antigens described herein or known in the art. In some embodiments, the tumor antigen or cell surface molecule is a polypeptide. In some embodiments, the tumor antigen or cell surface molecule is selectively expressed or overexpressed on tumor cells as compared to non-tumor cells of the same tissue. In some embodiments, the CAR binds a pathogen-specific antigen. In some embodiments, the CAR is specific for viral antigens (such as HIV, HCV, HBV, etc.), bacterial antigens and/or parasitic antigens.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions include those derived from (i.e., include at least the transmembrane region(s) of) the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154. Alternatively, the transmembrane domain may be synthetic and include predominantly hydrophobic residues such as leucine and valine, and further include a triplet of phenylalanine, tryptophan and valine at each end of the synthetic transmembrane domain.

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The CAR generally includes an intracellular signaling component or components. Ideally, the CAR includes an intracellular component of the TCR complex, such as a TCR CD3⁺ chain that mediates T cell activation and cytotoxicity, e.g., CD3 zeta chain. Thus, in some aspects, the antigen binding molecule is linked to one or more cell signaling modules. In some embodiments, cell signaling modules include CD3 transmembrane domain, CD3 intracellular signaling domains, and/or other CD transmembrane domains. In some embodiments, the CAR further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor γ and CD8, CD4, CD25 or CD16.

Upon ligation of the CAR, the cytoplasmic domain or intracellular signaling domain of the CAR activates at least one of the normal effector functions of the immune cell, e.g., the T cell engineered to express the CAR. For example, in some contexts, the CAR induces a function of a T cell such as cytolytic activity or T-helper activity, such as secretion of cytokines or other factors. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

Primary cytoplasmic signaling sequences can in some aspects regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, DAP10, and ICOS.

CARs and the production and introduction thereof into cells can be carried out as described, for example, in U.S. Pat. Nos. 6,451,995; 7,446,190; 8,252,592; EP 2537416; and WO 2013/126726; and/or by Sadelain, et al. (2013) Cancer Discov. 3(4):388-398; Davila, et al. (2013) PLoS ONE 8(4):e61338; Turtle, et al. (2012) Curr. Opin. Immunol. 24(5): 633-39; or Wu, et al. (2012) Cancer March 18(2):160-75.

In addition to CARs, any other genetic modification could be introduced into the CD7-negative, CD3-positive T cell population. This includes, but is not limited to, recombinant T cell receptors, antibodies, bispecific antibodies, cytokines, cytokine receptors, chimeric cytokine receptors, safety switches. Genetic modifications also include gene editing (for example CRISPR/Cas9, TALENs) and silencing of gene expression using shRNA technologies as described further herein.

In some embodiments, the stimulated CD7⁻ CD3⁺ T cells are modified with a recombinant T cell receptor (TCR). In some embodiments, the recombinant TCR is specific for an antigen, generally an antigen present on a target cell, such as a tumor-specific antigen, an antigen expressed on a particular cell type associated with an autoimmune or inflammatory disease, or an antigen derived from a viral pathogen or a bacterial pathogen.

In alternative embodiments, the stimulated CD7⁻ CD3⁺ T cells are engineered to express T cell receptors cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., Parkhurst, et al. (2009) Clin. Cancer Res. 15:169-180 and Cohen, et al. (2005) J. Immunol. 175:5799-5808. In some embodiments, phage display is used to isolate TCRs against a target antigen (see, e.g., Varela-Rohena, et al. (2008) Nat. Med. 14:1390-1395 and Li (2005) Nat. Biotechnol. 23:349-354.

After the T cell clone is obtained, the TCR alpha and beta chains are isolated and cloned into a gene expression vector. The TCR alpha and beta genes may be linked via a picornavirus 2A ribosomal skip peptide so that both chains are co-expressed. In some embodiments, genetic transfer of the TCR is accomplished via retroviral or lentiviral vectors, or via transposons by known methods. See, e.g., Baum, et al. (2006) Mol. Ther. 13:1050-1063; Frecha, et al. (2010) Mol. Ther. 18:1748-1757; and Hackett, et al. (2010) Mol. Ther. 18:674-683.

Overexpression of a stimulatory factor (for example, a lymphokine or a cytokine) may be toxic to a subject. Thus, in some contexts, the engineered CD7⁻ CD3⁺ T cells include gene segments that cause the cells to be susceptible to negative selection in vivo, such as upon administration in adoptive immunotherapy. For example, in some aspects, the CD7⁻ CD3⁺ T cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler, et al. (1977) Cell 11:223-232), which confers ganciclovir sensitivity; the cellular hypoxanthine phosphoribosyltransferase (HPRT) gene, the cellular adenine phosphoribosyltransferase (APRT) gene, or bacterial cytosine deaminase (Mullen, et al. (1992) Proc. Natl. Acad. Sci. USA 89:33).

In some aspects, the CD7⁻ CD3⁺ T cells further are engineered to promote expression of cytokines, such as proinflammatory cytokines, e.g., IL-2, IL-12, IL-7, IL-15, IL-21.

Various methods for the introduction of genetically engineered components, e.g., antigen receptors such as CARs, are well known and may be used with the methods and compositions herein. Exemplary methods include those for transfer of nucleic acids encoding the receptors, including via viral, e.g., retroviral or lentiviral, transduction, transposons, and electroporation.

In some embodiments, recombinant nucleic acids are introduced into cells using recombinant infectious virus particles, such as, e.g., vectors derived from simian virus (SV40), adenoviruses, adeno-associated virus (AAV). In other embodiments, recombinant nucleic acids are introduced into the CD7⁻ CD3⁺ T cells using recombinant lentiviral vectors or retroviral vectors, such as gamma-retroviral vectors (see, e.g., Koste, et al. (2014) Gene Therapy doi: 10.1038/gt.2014.25; Carlens, et al. (2000) Exp. Hematol. 28(10):1137-46; Alonso-Camino, et al. (2013) Mol. Ther. Nucl. Acids 2:e93; Park, et al. (2011) Trends Biotechnol. 29(11):550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller & Rosman (1989) BioTechniques 7:980-990; Miller (1990) Human Gene Therapy 1:5-14; Scarpa, et al. (1991) Virology 180:849-852; Burns, et al. (1993) Proc. Natl. Acad. Sci. USA 90:8033-8037; and Boris-Lawrie & Temin (1993) Cur. Opin. Genet. Develop. 3:102-109.

Methods of lentiviral transduction are known. Exemplary methods are described in, e.g., Wang, et al. (2012) J. Immunother. 35(9):689-701; Cooper, et al. (2003) Blood. 101:1637-1644; Verhoeyen, et al. (2009) Methods Mol Biol. 506:97-114; and Cavalieri, et al. (2003) Blood. 102(2):497-505.

In some embodiments, recombinant nucleic acids are introduced into CD7⁻ CD3⁺ T cells via electroporation (see, e.g., Chicaybam, et al. (2013) PLoS ONE 8(3):e60298 and Van Tedeloo, et al. (2000) Gene Therapy 7(16):1431-1437). In some embodiments, recombinant nucleic acids are transferred into the CD7⁻ CD3⁺ T cells via transposition (see, e.g., Manuri, et al. (2010) Hum. Gene Ther. 21(4):427-437; Sharma, et al. (2013) Molec. Ther. Nucl. Acids 2:e74; and Huang, et al. (2009) Methods Mol. Biol. 506:115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston (1990) Nature 346:776-777 (1990)); and strontium phosphate DNA co-precipitation (Brash, et al. (1987) Mol. Cell Biol. 7:2031-2034).

In some aspects, the selecting, incubating, and/or engineering steps are carried out in a sterile or contained environment and/or in an automated fashion, such as controlled by a computer attached to a device in which the steps are performed.

In some embodiments, the provided methods include steps for freezing, e.g., cryopreserving, the cells, either before or after selection, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

Also provided are kits useful in performing the provided methods. In some embodiments, the kits include antibodies, generally coupled to solid supports, for the isolation, e.g., for immunoaffinity-based separation steps, of the methods. In some embodiments, the kit includes anti-CD7 and anti-CD3 antibodies for negative and positive selection, respectively, bound to magnetic beads. In one embodiment, the kit includes instructions to carry out selection starting with a sample, such as a PBMC sample, by selecting CD7⁺ cells, recognized by the anti-CD7 antibodies provided with the kit, retaining both positive and negative fractions. In some aspects, the instructions further include instructions to carry out the additional selection of CD3⁺ cells, starting with the CD7-negative fraction, while maintaining the compositions in a contained environment. In some embodiments, the kit further includes instructions to transfer the cells of the populations isolated by the selection steps to a culture, cultivation, or processing vessel. In an alternative embodiment, the kit includes a vector harboring nucleic acids encoding a CAR and instructions for introducing the vector into the CD7⁻ CD3⁺ cells.

Also provided are cells, cell populations, and compositions (including pharmaceutical and therapeutic compositions) containing the cells and populations, produced by the provided methods. Also provided are methods, e.g., therapeutic methods for administrating the cells and compositions to subjects, e.g., patients.

Provided are methods of administering the cells, populations, and compositions, and uses of such cells, populations, and compositions to treat or prevent diseases, conditions, and disorders, including cancers. In some embodiments, the cells, populations, and compositions are administered to a subject or patient having the particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

Methods for administration of cells for adoptive cell therapy are known and may be used in connection with the provided methods and compositions. For example, adoptive T cell therapy methods are described, e.g., in US 2003/0170238; U.S. Pat. No. 4,690,915; Rosenberg (2011) Nat. Rev. Clin. Oncol. 8(10):577-85). See also Themeli, et al. (2013) Nat. Biotechnol. 31(10):928-933; Tsukahara, et al. (2013) Biochem. Biophys. Res. Commun. 438(1):84-9; Davila, et al. (2013) PLoS ONE 8(4):e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

Among the diseases, conditions, and disorders for treatment with the provided compositions, cells, methods and uses are tumors, including solid tumors, hematologic malignancies, and melanomas, and infectious diseases, such as infection with a virus or other pathogen, e.g., HIV, HCV, HBV, CMV, and parasitic disease. In some embodiments, the disease or condition is a tumor, cancer, malignancy, neoplasm, or other proliferative disease. Such diseases include but are not limited to leukemia, lymphoma, e.g., chronic lymphocytic leukemia (CLL), ALL, non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin (including melanoma), bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma. In particular embodiments, the cancer is CD7⁺ cancer, such as CD7⁺ leukemia.

In some embodiments, the disease or condition is an infectious disease or condition, such as, but not limited to, viral, retroviral, bacterial, and protozoal infections, immunodeficiency, Cytomegalovirus (CMV), Epstein-Ban virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant.

In some embodiments, the antigen associated with the disease or disorder is selected from the group of orphan tyrosine kinase receptor ROR1, EGFR, Her2, L1-CAM, CD7, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acetylcholine receptor, GD2, GD3, HMW-MAA, IL-22R-alpha, IL-13R-alpha, kappa light chain, Lewis Y, MAGE-A1, MAGE-A3, mesothelin, MUC1, MUC16, PSMA, PSCA, NKG2D ligands, NY-ESO-1, MART-1, gp100, oncofetal antigen, TAG72, VEGFR2, estrogen receptor, progesterone receptor, CD123, CS-1, c-Met, and/or molecules expressed by HIV, HCV, HBV or other pathogens.

In some embodiments, the cells and compositions are administered to a subject in the form of a pharmaceutical composition, such as a composition including the cells or cell populations and a pharmaceutically acceptable carrier or excipient. The pharmaceutical compositions in some embodiments additionally include other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluene sulphonic acid.

The choice of carrier will in the pharmaceutical composition is determined in part by the particular engineered CAR or TCR, vector, or cells expressing the CAR or TCR, as well as by the particular method used to administer the vector or host cells expressing the CAR. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

In addition, buffering agents may be included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known.

Exemplary methods are described in more detail in, for example, Remington: The Science and Practice of Pharmacy, Lippincott Williams & Wilkins; 21^{st} ed. (May 1, 2005).

Ideally, the pharmaceutical composition includes the cells or cell populations in an amount that is effective to treat or prevent the disease or condition, such as a therapeutically effective or prophylactically effective amount. Thus, the methods of treatment include administration of the cells and populations at effective amounts. Therapeutic or prophylactic efficacy may be monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered at a desired dosage, which in some aspects includes a desired dose or number of cells or cell type(s). Thus, the dosage of cells in some embodiments is based on a total number of cells (or number per kg body weight). In some aspects, the desired dose is at or above a minimum number of cells or minimum number of cells per unit of body weight. In certain embodiments, the cells or population of cells, are administered to the subject at a range of about one million to about 100 billion cells, such as, e.g., 1 million to about 50 billion cells, or a range defined by any two of the foregoing values. In some embodiments, the dose of total cells and/or dose of population of cells is within a range of between at or about 10⁴ and at or about 10⁹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight.

The cell populations and compositions in some embodiments are administered to a subject using standard administration techniques, including oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

The cell populations obtained using the methods described herein may be co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cell populations are administered prior to the one or more additional therapeutic agents. In some embodiments, the cell populations are administered after to the one or more additional therapeutic agents.

Following administration of the cells, the biological activity of the engineered cell populations may be measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In addition, the ability of the engineered cells to destroy target cells can be measured using any suitable method known in the art, such as cytotoxicity assays described in, for example, Kochenderfer, et al. (2009) J. Immunotherapy 32(7):689-702, and Herman, et al. (2004) J. Immunological Methods 285(1):25-40. In some embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

The following non-limiting examples are provided to further illustrate the present invention.

### Example 1: Isolation of CD7⁻ CD3⁺ T Cells

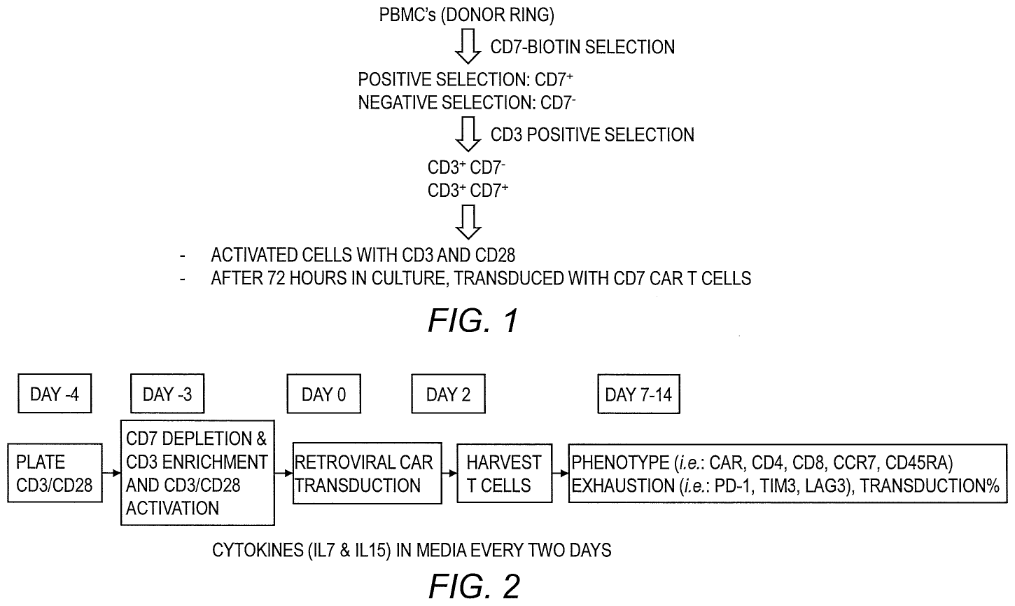

Overview. CD7⁻ T cells were isolated from PBMCs using a 2-step magnetic bead depletion/selection procedure (CD7 depletion followed by selection of CD3⁺ T cells from the CD7⁻ fraction; FIG. 1). The methodology for preparing and characterizing the CD7⁻ T cells is depicted in FIG. 2 and described in detail below. Non-selected T cells (bulk T cells), CD7⁺ and CD7⁻ T cells were activated and transduced with a retroviral vector encoding a CD7 CAR with a CD28.z signaling domain, and expanded with IL7/1L15. The effector function of CD7⁻ T cells expressing CD7 CARs (CD7 CAR^{CD7-} T cells) was assessed in vitro as well as in xenograft models.

Day −4. Four days prior to retroviral transduction, anti-CD3 (Miltenyi) and anti-CD28 (Miltenyi) antibodies were diluted to a final concentration of 0.5 μg/mL in sterile water. To each well of a non-tissue culture treated, 24-well plate (Thermo) was added 0.5 mL (25 μg) of each antibody. The plate was sealed and incubated at +4° C. overnight.

Day −3. Three days prior to retroviral transduction, CD7⁺ cells were depleted and CD3⁺ cells were enriched for. In particular, frozen PBMCs (e.g., 25×10⁷ cells) were thawed in a water bath, resuspended in warm complete media (RPMI 1640 with 10% FBS and 1% GlutaMAX) and subsequently centrifuged at 400 g for 5 minutes. The supernatant was aspirated and the pellet was resuspended in cell sorting buffer (25 ml BSA stock solution (Miltenyi) and 2 ml 0.5M EDTA pH8.0 (Life Technology) in 500 ml 1×PBS). Cells were counted and subsequently centrifuged at 400 g for 5 minutes. The medium was aspirated and the pellet was resuspended in 500 μL of cell sorting buffer.

For CD7⁺ cell depletion, 10 μL of anti-CD7-Biotin antibody (Miltenyi) and 10 μL of FcR Blocking Reagent were added to 10⁷ cells. The mixture was incubated for 10 minutes at 4° C. The cells were washed with 10 volumes of cell sorting buffer and subsequently centrifuged at 400 g for 5 minutes. To the pellet of cells was added 20 μL of anti-biotin microbeads (Miltenyi) per 10⁷ cells. The cells were mixed well and incubated for 15 minutes at 4° C. The cells were washed with 25 mL of cell sorting buffer and subsequently centrifuged at 400 g for 5 minutes. During the wash steps, the magnetic-activated cell sorting column (LS column, Miltenyi) was equilibrated by placing the column in a magnetic holder, rinsing the column with 3 mL of cell sorting buffer, and discarding the flow-through.

After centrifugation of the anti-biotin microbead-treated cells, the supernatant was aspirated and the cells were resuspended in 500 μL of cell sorting buffer. The cells were subsequently passed through a 40 μm cell strainer to remove dead cell clumps. The cell suspension was applied to the magnetic-activated cell sorting column and the cells were allowed to enter the column. The column was washed three times with 3 mL of cell sorting buffer. The flow-through cells were collected in a fresh 15 ml tube and labelled as “CD7-ve cells.” The column was removed from the magnetic holder, 5 mL of cell sorting buffer was applied to the column and the remaining cells were collected in a new 15 mL tube. These cells were labelled as “CD7⁺ ve cells.” All cells were washed with fresh media and subsequently centrifuged at 400 g for 5 minutes.

For CD3⁺ cell enrichment, the CD7-ve cells were washed with 25 mL of cell sorting buffer and subsequently centrifuged at 400 g for 5 minutes. To the cells was added 20 μL of anti-CD3 microbeads (Miltenyi) per 10⁷ cells. The cells were mixed well and incubate for 30 minutes at 4° C. The cells were washed with 20 mL of cell sorting buffer and subsequently centrifuged at 400 g for 5 minutes. As above, the LS column was equilibrated with cell sorting buffer during the cell washing step.

After centrifugation of the anti-CD3 microbead-treated cells, the supernatant was aspirated and the cells were resuspended in 500 μL of cell sorting buffer. The cells were subsequently passed through a 40 μm cell strainer to remove dead cell clumps. The cell suspension was applied to the magnetic-activated cell sorting column and the cells were allowed to enter the column. The column was washed three times with 3 mL of cell sorting buffer. The flow-through cells were collected in a fresh 15 ml tube and labelled as “CD3− CD7− cells.” The CD3+ cells were collected by removing the column from the magnetic holder, adding 5 mL of cell sorting buffer to the column and collecting the cells in a new 15 mL tube. These cells were labelled as “CD3+CD7− cells.” These CD3+CD7− cells were centrifuged at 400 g for 5 minutes and subsequently resuspended in 5 mL of complete media for cell counting.

CD3+CD7− cells were centrifuged and resuspended at 1×10⁶ cells/ml in complete media containing cytokines (IL-7 at 10 ng/mL and IL-15 at 10 ng/mL). The antibody solution applied to the non-tissue culture plate on day −4 was aspirated and the wells were washed with complete media. To each well of the plate was added 1 mL of the CD3+CD7− cells (1×10⁶ cells/well) and 1 mL of fresh media with cytokines.

Day −1. One day prior to retroviral transduction, wells of the non-tissue culture treated 24-well plate were coated with retronectin (10 μg/well in 500 ml of 1×PBS) and the plate was sealed. The retronectin-coated plate was incubated at +4° C. overnight.

Day 0. On the day of retroviral transduction, the retronectin solution was removed from the wells of the plate and the wells were washed once with RPMI complete media. To each well of the plate was added 500 μL of viral supernatant and the plate was centrifuged at 2000 g for 90 minutes. Blasts were prepared at 1.25×10⁵ cells/cc with IL-7 at 10 ng/mL and IL-15 at 5 ng/mL. Upon removal of the viral supernatant, 2 mL of the blasts (0.25×10⁶ cells) were added to each well.

Day 2. Two days after retroviral transduction, 1 mL of medium was removed from the wells and the cells were transferred to a new plate. To the wells of the new plate was added 1 mL of complete media containing IL-7 at 10 ng/mL and IL-15 at 5 ng/ml. Every two days, the cells were feed with additional IL-7 at 10 ng/mL and IL-15 at 5 ng/mL. The resulting cells were either frozen or used in experiments within 7 to 14 days of transduction.

### Example 2: Characterization and Use of CD7⁻ T Cells

Cells and Culture Conditions. De-identified apheresis products from healthy donors were purchased from Key Biologics (Memphis, Tenn.) or obtained through a deidentified product protocol and were used in accordance with the Helsinki Declaration. Authenticated K562, CCRF, Daudi and Molm13 cell lines were obtained from ATCC. Molm13, CCRF and Daudi expressing a GFP firefly luciferase (Molm13.ffluc) fusion molecule were previously described (e.g., Bonifant, et al. (2016) Mol. Ther. 24(9):1615-26). All cell lines were cultured in RPMI media (GE Healthcare Life Sciences; Logan, Utah) supplemented with 10% fetal bovine serum (Gibco/Thermo Fisher Scientific, Waltham, Mass.) and L-glutamine (GlutaMAX; Gibco/Thermo Fisher Scientific).

Generation of Retroviral Vectors. The generation of SFG retroviral vectors encoding the CD19 or CD7 CARs were previously described (e.g., Velasquez, et al. (2016) Sci. Rep. 6:27130; Gomes-Silva, et al. (2017) Blood 130(3):285-296). RD114-pseudotyped retroviral particles were generated as previously described (Chow (2013) Mol. Ther. 21:629-37).

Flow Cytometry. Cells were stained with fluorochrome-conjugated primary antibodies for 30 minutes at 4° C. and washed with FACS buffer (2% FBS in 1×PBS) prior to analysis. For CAR staining, cells were washed with 1×PBS twice then incubated with a CD7 antibody in PBS for 30 minutes at 4° C. and washed with FACS buffer prior to analysis. Stained cells were run on a BD FACSLyric system (BD Biosciences) and analysis was done using FlowJo software. The following antibodies were used: CD4 (Clone OKT4; BV785, BioLegend); CD8 (Clone SK1; APC-Cy7; BD Pharmingen); CCR7 (Clone REA546; PE; Miltenyi); CD45RO (Clone UCHL1; APC; Tonbo); Tim3 (Clone F38-2E2; PE-Cy7; Biolegend); PD1 (Clone EH12.2H7; BV421; Biolegend); Goat anti-human Fc-IgG (pooled goat antisera; PE; Southern Biotech).

Cytotoxicity Assay. Cytotoxic activity was evaluated using a luciferase assay. Target cells which were used expressed firefly luciferase (CCRF.ffluc, Daudi.ffluc). Target cells were incubated overnight either alone or with effector T cells in round bottom 96-well plates (Corning; Corning, N.Y.) at effector to target ratios (E:T) of 1:1, 1:2,1:4,1:8,1:16,1:32,1:64. Luciferase activity was determined according to the manufacturer's instructions using a luciferase assay kit (Promega, Madison, Wis.) and a multimode plate reader (Tecan, Maennedorf, Switzerland).

Cytokine Production. Effector cells were cultured at a 2:1 ratio with target cells or in the presence of media alone for 24 hours in a 24-well plate (Corning). Supernatant was collected and IFNγ was determined using an ELISA kit (R&D; Minneapolis, Minn.), according to manufacturer's instructions.

Xenograft Model. In vivo experiments were performed under a protocol approved by SJCRH Institutional Animal Care and Use Committee (IACUC). Animals were housed in specific pathogen-free rooms for the duration of the experiments. Female NSG mice (NOD-scid IL2Rgammam^{null}, NOD-scid IL2Rg^{null}, NSG, NOD-scid gamma) were obtained from the SJCRH breeding colony at 8-10 weeks of age. Mice received 5×10³ Molm13 tumor cells modified to express a GFP.ffluc fusion gene (Molm13.ffluc) i.v. (tail vein injection). Seven days later, mice in the treatment groups were infused with effector cells. Animals receiving tumor only were used as controls. Serial imaging was performed subsequently. The mice were imaged at the SJCRH Center for In Vivo Imaging and Therapeutics using the Xenogen IVIS®-200 imaging system (IVIS, Xenogen Corp., Alameda, Calif.) and euthanized at predefined endpoints or when they met euthanasia criteria in accordance with SJCRH's Animal Resource Center.

Statistics. Data were summarized using descriptive statistics. Comparisons of continuous variables among three or more groups were made by one-way ANOVA, while comparisons between two groups were made by t test or Wilcoxon rank-sum test when appropriate. Multiple comparisons were adjusted by the Holm's method. Survival times from tumor cell injection in the mouse experiments were analyzed by the Kaplan-Meier method and the Gehan-Wilcoxon test. GraphPad Prism 8 software (GraphPad software), and R 3.6.0 (Lucent technologies, Murray Hill, New Providence, N.J.) were used for statistical analysis. P values <0.05 were considered statistically significant.

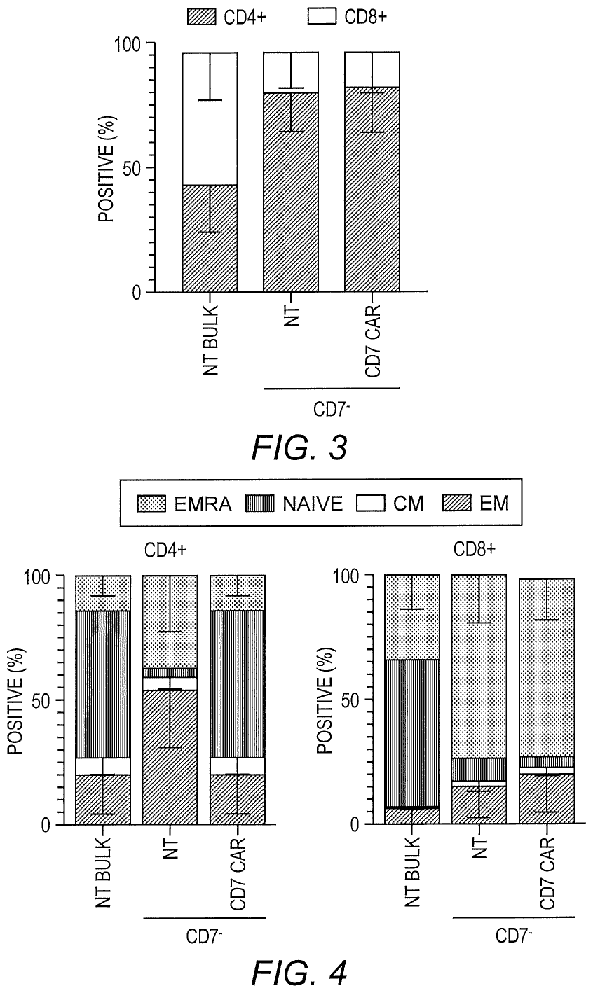

Results. To assess the feasibility of isolating CD7⁻ T cells, the frequency of CD7⁻ T cells in PBMCs was determined. On average, 4.7% of T cells were CD7⁻ (range: 2% to 12.3%; N=25; FIG. 3). Notably, using the combined CD7 depletion/CD3 selection method of the invention, these cells were successfully selected from bulk PBMCs.

To assess the use of the cells of the invention, CD7⁻, CD7⁺ and bulk T cells were genetically modified to express a CD7 CAR (CD7 CAR^{CD7-}, CD7 CAR^{CD7+}, CD7 CAR^{Bulk}). The CD7 CAR construct was composed of a CD7 scFv cloned into a second-generation backbone CAR containing CD28 and CD3z endodomains and the CH3 domain from IgG1 as a spacer region. Transduction efficiencies ranged from 31% to 66% (±5%) for each T cell population were obtained. In particular, the transduction efficiency of CD7 CAR^{CD7-} cells was 56±17% (N=9). Post-transduction, CD7 CAR^{CD7-} T cells did not undergo fratricide and had similar expansion kinetics (N=9, p=ns) in comparison to non-transduced (NT) T cell cultures (NT CD7⁻, NT CD7⁺, NT bulk). In contrast, CD7 CAR^{CD7+} and CD7 CAR^{Bulk} T cells underwent fratricide and did not expand (N=9 P=0.0003), which also resulted in a decrease in viability when compared to CD7 CAR^{CD7-} and NT (N=9 P=0.0024).

Flow cytometry was used to stain T cells for CD4 and CD8 populations. NT^{CD7-} and CD7 CAR^{CD7-} T cells were composed of a predominant CD4+ population (N=8) on day 7 (FIG. 3) and day 14 of culture. Further, CCR7 and CD45RA expression was used to phenotype T cells as Naïve (CCR7⁺/CD45RA⁺), Central memory (CCR7⁺/CD45RA⁻), effector memory (CCR7⁻/CD45RA⁻), and terminal effector (CCR7⁻/CD45RA⁺). This analysis indicated that CD7⁻ T cells (NT^{CD7-} and CD7 CAR^{CD7-} T cells) were predominantly memory phenotype as determined on day 7 and 14 of culture (FIG. 4).

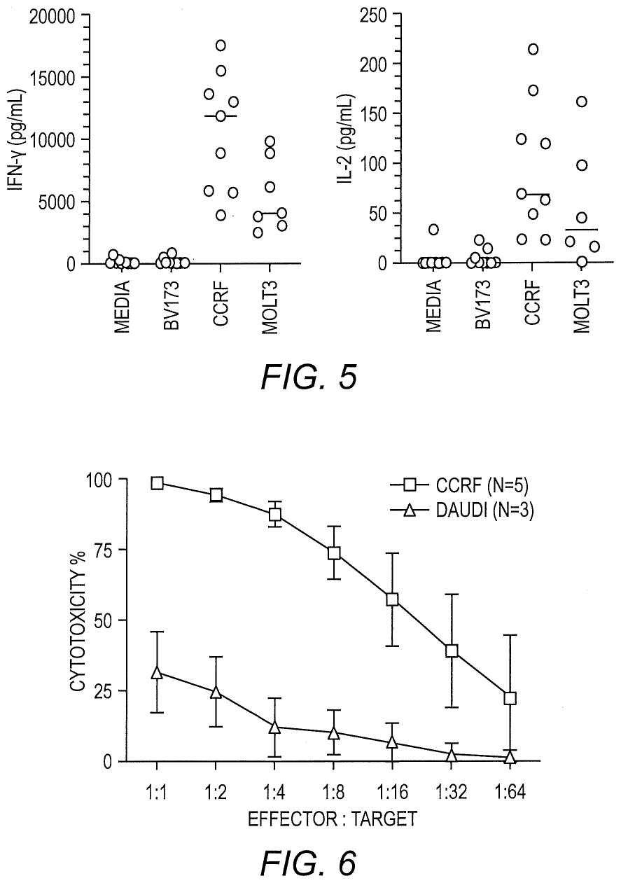

To assess the effector function of CD7 CAR^{CD7-} T cells, CD7⁺ T-ALL cell lines (CCRF, MOLT3) were analyzed. CD7 CAR^{CD7-} T cells recognized CD7⁺ targets in contrast to CD7⁻ targets (BV173, Daudi) as evidenced by significant (N=6, p<0.0001) IL-2 production and IFN-γ production (FIG. 5). Control CAR T cells (CD19 CAR^{CD7-}) did not recognize CD7⁺ target cells, confirming specificity. CD7 CAR^{CD7-} T cells also had potent cytolytic activity against CD7⁺ targets in cytotoxicity assays. In particular, CD7 CAR^{CD7-} T cells were incubated with increasing amounts of luciferase-tagged tumor cells (CCRF.ffluc or Daudi.ffluc) for 24 hours. Tumor cell lysis was assessed with a luciferase-based cytotoxicity assay. CD7 CAR^{CD7-} T cells lysed CD7⁺ (CCRF) but not CD7⁻ (Daudi.ffluc) targets (N=3 P=0.0025)(FIG. 6).

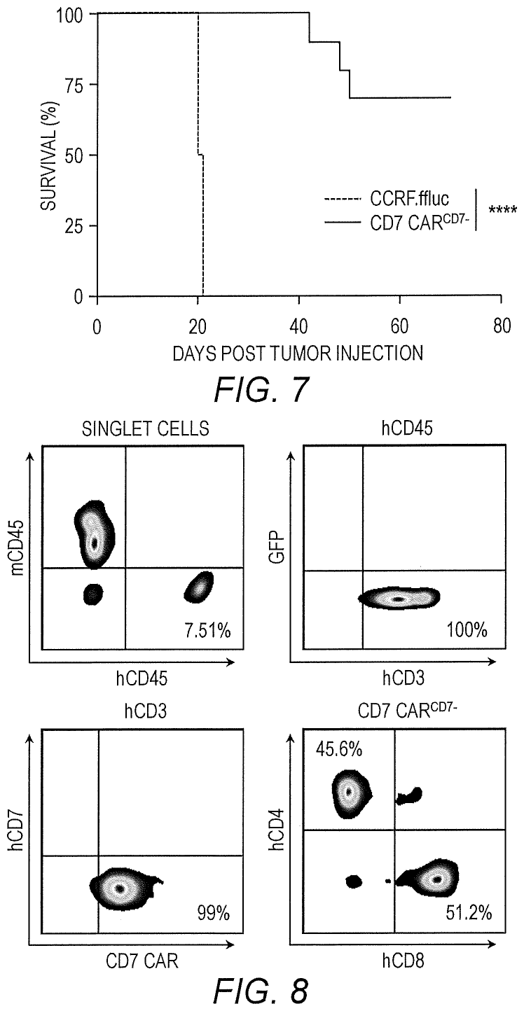

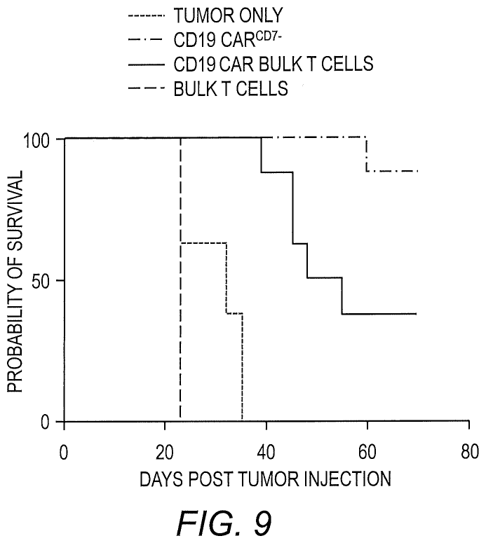

To assess in vivo anti-tumor activity of CD7 CAR^{CD7-} T cells, an NSG mouse xenograft model with CCRF cells genetically modified to express firefly luciferase (CCRF.ffluc) were used thereby allowing for serial bioluminescence imaging. In particular, NSG mice (N=10 per group) were injected intravenously with 1×10⁴ CCRF cells labeled with firefly luciferase. One week later, mice were injected with 1×10⁷ CD7 CAR^{CD7-} T cells for the treatment group only. T-ALL progression was tracked by bioluminescence imaging. Animals receiving only tumor cells had progression of disease and required euthanasia around day 20. By comparison, a single infusion of CD7 CAR^{CD7-} T cells had potent anti-leukemia activity as judged by serial imaging resulting in a significant survival (p<0.003) advantage in comparison to control mice (FIG. 7). Peripheral blood evaluation by flow cytometry demonstrated persistence of the CD7 CAR⁺ T cells (FIG. 8). Similar results have been obtained when the CD7-negative T cells are transduced with a CD19 CAR (FIG. 9).

What is claimed is:

1. A method for preparing a population of CD7-negative, CD3-positive T cells comprising
(a) performing a first selection comprising depleting, from a population of primary immune cells, cells that express CD7 thereby generating a population of CD7-negative cells;
(b) performing a second selection comprising enriching, from the population of CD7-negative cells, T cells that express CD3 thereby generating a population of CD7-negative and CD3-positive T cells, and
(c) incubating the population of CD7-negative and CD3-positive T cells in a culture vessel under stimulating conditions, thereby generating stimulated CD7-negative, CD3-positive T cells.

2. The method of claim 1, further comprising genetically modifying the stimulated T cells.

3. A method for preparing genetically modified CD7-negative, CD3-positive T cells comprising
(a) performing a first selection comprising depleting, from a population of primary immune cells, cells that express CD7 thereby generating a population of CD7-negative cells;
(b) performing a second selection comprising enriching, from the population of CD7-negative cells, T cells that express CD3 thereby generating a population of CD7-negative and CD3-positive T cells,
(c) incubating the population of CD7-negative and CD3-positive T cells in a culture vessel under stimulating conditions, thereby generating stimulated T cells; and
(d) genetically modifying the stimulated T cells generated in (c), thereby preparing genetically modified CD7-negative, CD3-positive T cells.

4. The method of claim 3, wherein the first, second, or first and second selection comprises immunoaffinity-based selection.

5. The method of claim 4, wherein the immunoaffinity-based selection comprises an antibody immobilized on or attached to an affinity chromatography matrix, magnetic particle or label.

6. The method of claim 3, wherein the stimulated T cells are genetically modified by introducing nucleic acids encoding a genetically engineered antigen receptor into the stimulated T cells.

7. A method for preparing CD7-negative, CD3-positive, chimeric antigen receptor T cells comprising
(a) performing a first selection comprising depleting, from a population of primary immune cells, cells that express CD7 by
(i) contacting the population of primary immune cells with a first immunoaffinity reagent that specifically binds to CD7 on the surface of the primary immune cells; and
(ii) recovering cells of the population that are not bound to the first immunoaffinity reagent thereby generating a population of CD7-negative cells;
(b) performing a second selection comprising enriching, from the population of CD7-negative cells, T cells that express CD3 by
(i) contacting the population of CD7-negative cells with a second immunoaffinity reagent that specifically binds to CD3 on the surface of CD7-negative cells;
(ii) recovering T cells of the population that are bound to the second immunoaffinity reagent thereby generating a population of CD7-negative and CD3-positive T cells,
(c) incubating the population of CD7-negative and CD3-positive T cells in a culture vessel under stimulating conditions, thereby generating stimulated T cells; and
(d) introducing a genetically engineered antigen receptor into the stimulated T cells generated in (c),
thereby preparing CD7-negative, CD3-positive, chimeric antigen receptor T cells.

8. A population of CD7-negative, CD3-positive T cells prepared by the method of claim 1.

9. A population of genetically modified CD7-negative, CD3-positive T cells CD7-negative, CD3-positive T cells prepared by the method of claim 3.

10. A population of CD7-negative, CD3-positive, chimeric antigen receptor T cells prepared by the method of claim 7.

11. A method for treating cancer comprising delivering to a subject in need of treatment an effective amount of the CD7-negative, CD3-positive, chimeric antigen receptor T cells of claim 10 thereby treating the subject's cancer.

12. The method of claim 11, wherein the cancer is a CD7+ cancer.
